(19) United States
(12) Patent Application Publication
(10) Pub. No.: US 2025/0127626 A1
(43) Pub. Date: Apr. 24, 2025

(54) INTERBODY SPACER AND BONE PLATE ASSEMBLY

(71) Applicant: Lincotek Medical LLC, Bartlett, TN (US)

(72) Inventors: Troy WALTERS, Providence, UT (US); Trevor LEWIS, Lehi, UT (US)

(21) Appl. No.: 18/919,185

(22) Filed: Oct. 17, 2024

Related U.S. Application Data
(60) Provisional application No. 63/544,773, filed on Oct. 18, 2023.

(51) Int. Cl. A61F 2/44; A61B 17/80; A61F 2/30

(52) U.S. Cl. CPC A61F 2/4455; A61B 17/80; A61F 2/30724

(57) ABSTRACT

An implant assembly for stabilizing a joint between a superior vertebra and an inferior vertebra may include a bone plate and an interbody spacer. The bone plate may include an anterior member having at least one superior fastener aperture and at least one inferior fastener aperture formed therethrough. The bone plate may also include a first arm extending from a first end of the anterior member, and a second arm extending from a second end of the anterior member, opposite the first arm, forming a cavity therebetween. The interbody spacer may be configured for placement within the cavity of the bone plate. At least a portion of the bone plate may be formed via at least one subtractive manufacturing process, and at least a portion of the interbody spacer may be formed via at least one additive manufacturing process.

### CROSS-REFERENCE TO RELATED APPLICATIONS

This application claims the benefit of U.S. Provisional Patent Application Ser. No. 63/544,773 filed on Oct. 18, 2023 and entitled “INTERBODY SPACER AND BONE PLATE ASSEMBLY”. The above-referenced document is hereby incorporated by reference in its entirety.

### TECHNICAL FIELD

The present disclosure relates to surgical devices, systems, methods, and instruments. More specifically, the present disclosure relates to orthopedic fixation devices, systems, methods, and instruments utilizing a bone plate coupled to an interbody spacer and at least one fastener for stabilizing a joint between a superior vertebra and an inferior vertebra.

### BACKGROUND

Spinal fixation implants are often used to immobilize, stabilize, and/or fuse spinal joints between a superior vertebra and an inferior vertebra. Spinal fixation implants may speed up/promote bone fusion of spinal joints to treat spinal deformities and instabilities in the cervical, thoracic, lumbar, and/or sacral regions of the spine. Example spinal maladies that may benefit from spinal fusion include spondylolisthesis, degenerative disc disease, trauma, scoliosis, kyphosis, lordosis, spinal stenosis, spinal tumors, pseudoarthrosis, revision fusion surgeries, etc.

Interbody spacers may be implanted in patients via surgical techniques such as Anterior Lumbar Interbody Fusion (ALIF), Posterior Lumbar Interbody Fusion (PLIF), Transforaminal Lumbar Interbody Fusion (TLIF), etc. Interbody spacers may be placed within an intervertebral space between adjacent vertebrae in the spine and an exterior bone plate may also be implanted therebetween to help stabilize adjacent vertebrae during the bone fusion process. The interbody spacers and/or associated exterior bone plates should have sufficient structural integrity to withstand the stress of maintaining an intervertebral space and staying firmly in place during the bone fusion process.

Accordingly, robust and efficient spinal fixation implants, systems, methods, and instruments that can help reduce risk, promote better outcomes for patients, decrease costs, and increase surgical procedure efficiency would be desirable.

### SUMMARY

The orthopedic fixation devices, systems, and methods of the present disclosure have been developed in response to the present state of the art, and in particular, in response to the problems and needs in the art that have not yet been fully solved by currently available orthopedic fixation devices, systems, and methods. In some embodiments, the orthopedic fixation devices, systems, and methods of the present disclosure may provide improved devices, systems, and methods for stabilizing a joint between a superior vertebra and an inferior vertebra.

In some embodiments, an implant assembly for stabilizing a joint between a superior vertebra and an inferior vertebra may include a bone plate and an interbody spacer. The bone plate may include an anterior member having at least one superior fastener aperture formed therein and configured to receive at least one superior fastener therethrough, as well as at least one inferior fastener aperture formed therein and configured to receive at least one inferior fastener therethrough. The bone plate may also include a first arm extending from a first end of the anterior member, and a second arm extending from a second end of the anterior member, opposite the first arm, to form a cavity intermediate the first arm and the second arm of the bone plate, and the interbody spacer may be configured for placement within the cavity of the bone plate. At least a portion of the bone plate may be formed via at least one subtractive manufacturing process, and at least a portion of the interbody spacer may be formed via at least one additive manufacturing process.

In some embodiments of the implant assembly, the at least one additive manufacturing process may include at least one of: a 3D printing process, a porous surface deposition process, and a nano coating process.

In some embodiments of the implant assembly, the at least one subtractive manufacturing process may include at least one of: a machining process, a casting process, a molding process, a bone extraction process, and a bone shaping process.

In some embodiments of the implant assembly, the interbody spacer may include at least one porous region formed via a 3D printing process, and the bone plate may include at least one durable material formed via a machining process.

In some embodiments of the implant assembly, the interbody spacer may also include at least one solid region formed via the 3D printing process.

In some embodiments of the implant assembly, the interbody spacer may be configured to be inserted into the cavity of the bone plate from at least one of: a superior direction, an inferior direction, and a posterior direction.

In some embodiments of the implant assembly, the implant assembly may also include a locking plate that is couplable with the bone plate and configured to prevent the at least one superior fastener and the at least one inferior fastener from backing out of the implant assembly.

In some embodiments, an implant assembly for stabilizing a joint between a superior vertebra and an inferior vertebra may include a bone plate and an interbody spacer. The bone plate may include an anterior member having at least one superior fastener aperture formed therein and configured to receive at least one superior fastener therethrough, as well as at least one inferior fastener aperture formed therein and configured to receive at least one inferior fastener therethrough. The bone plate may also include a first arm extending from a first end of the anterior member, and a second arm extending from a second end of the anterior member, opposite the first arm, to form a cavity intermediate the first arm and the second arm of the bone plate. The interbody spacer may be configured for placement within the cavity of the bone plate. The interbody spacer may include at least one porous region formed via at least one additive manufacturing process, and at least one solid region formed via the at least one additive manufacturing process.

In some embodiments of the implant assembly, the at least one porous region may include a central porous region, and the at least one solid region may include a first solid region, a second solid region, and a third solid region.

In some embodiments of the implant assembly, the at least one additive manufacturing process may include at least one of: a 3D printing process, a porous surface deposition process, and a nano coating process.

In some embodiments of the implant assembly, at least a portion of the bone plate may be formed via at least one subtractive manufacturing process.

In some embodiments of the implant assembly, the at least one subtractive manufacturing process may include at least one of: a machining process, a casting process, a molding process, a bone extraction process, and a bone shaping process.

In some embodiments of the implant assembly, the interbody spacer may be configured to be inserted into the cavity of the bone plate from at least one of: a superior direction, an inferior direction, and a posterior direction.

In some embodiments of the implant assembly, the implant assembly may also include a locking plate couplable with the bone plate. The locking plate may be configured to prevent the at least one superior fastener and the at least one inferior fastener from backing out of the implant assembly.

In some embodiments, an inserter for implanting an implant assembly into an intervertebral joint space between a superior vertebra and an inferior vertebra may include a first inserter arm, a second inserter arm, and a pivot pin configured to pivotally couple the first inserter arm to the second inserter arm. The first inserter arm may include a proximal end, a distal end, and a first inserter arm connection feature disposed on the distal end of the first inserter arm. The second inserter arm may include a proximal end, a distal end, and a second inserter arm connection feature disposed on the distal end of the second inserter arm. In a first unlocked configuration, the proximal end of the first inserter arm may be pivoted away from the proximal end of the second inserter arm to pivot the distal end of the first inserter arm away from the distal end of the second inserter arm and receive the implant assembly between the first inserter arm connection feature and the second inserter arm connection feature. In a second locked configuration, the proximal end of the first inserter arm may be pivoted toward the proximal end of the second inserter arm to pivot the distal end of the first inserter arm toward the distal end of the second inserter arm and couple the implant assembly between the first inserter arm connection feature and the second inserter arm connection feature of the inserter.

In some embodiments of the inserter, the inserter may also include a rigid member formed on the first inserter arm and a resilient inserter member formed on the second inserter arm. The resilient inserter member may be configured to push against the rigid member to pivotally bias the proximal end of the first inserter arm away from the proximal end of the second inserter arm.

In some embodiments of the inserter, the inserter may also include a locking tab projecting from the first inserter arm, and a lock surface formed on a resilient locking member coupled to the second inserter arm. The lock surface formed on the resilient locking member may be configured to engage the locking tab projecting from the first inserter arm and hold the inserter in the second locked configuration.

In some embodiments of the inserter, the inserter may also include at least one inferior guide hole formed through at least one of the distal end of the first inserter arm and the distal end of the second inserter arm.

In some embodiments of the inserter, the inserter may also include at least one superior guide hole formed through at least one of the distal end of the first inserter arm and the distal end of the second inserter arm.

In some embodiments of the inserter, the at least one superior guide hole may include a first superior guide hole formed through the distal end of the first inserter arm, and a second superior guide hole formed through the distal end of the second inserter arm.

These and other features and advantages of the present disclosure will become more fully apparent from the following description and appended claims or may be learned by the practice of the orthopedic fixation devices, systems, and methods set forth hereinafter.

### BRIEF DESCRIPTION OF THE DRAWINGS

Exemplary embodiments of the present disclosure will become more fully apparent from the following description taken in conjunction with the accompanying drawings. Understanding that these drawings depict only exemplary embodiments and are, therefore, not to be considered limiting of the scope of the present disclosure, the exemplary embodiments of the present disclosure will be described with additional specificity and detail through use of the accompanying drawings in which:

FIG. 1 illustrates an exploded view of an implant assembly for stabilizing a spinal joint, according to an embodiment of the present disclosure;

FIG. 2 illustrates a perspective view of the implant assembly of FIG. 1, after assembly;

FIG. 3A illustrates a top perspective view of a bone plate, according to an embodiment of the present disclosure;

FIG. 3B illustrates a bottom perspective view of the bone plate of FIG. 3A;

FIG. 3C illustrates a front view of the bone plate of FIG. 3A;

FIG. 3D illustrates a rear view of the bone plate of FIG. 3A;

FIG. 3E illustrates a left side view of the bone plate of FIG. 3A;

FIG. 3F illustrates a right side view of the bone plate of FIG. 3A;

FIG. 3G illustrates a top view of the bone plate of FIG. 3A;

FIG. 3H illustrates a bottom view of the bone plate of FIG. 3A;

FIG. 4A illustrates a top perspective view of a spacer insert or solid regions of the interbody spacer, according to embodiments of the present disclosure;

FIG. 4B illustrates a bottom perspective view of the spacer insert or solid regions of the interbody spacer of FIG. 4A;

FIG. 4C illustrates a front view of the spacer insert or solid regions of the interbody spacer of FIG. 4A;

FIG. 4D illustrates a rear view of the spacer insert or solid regions of the interbody spacer of FIG. 4A;

FIG. 4E illustrates a left side view of the spacer insert or solid regions of the interbody spacer of FIG. 4A;

FIG. 4F illustrates a right side view of the spacer insert or solid regions of the interbody spacer of FIG. 4A;

FIG. 4G illustrates a top view of the spacer insert or solid regions of the interbody spacer of FIG. 4A;

FIG. 4H illustrates a bottom view of the spacer insert or solid regions of the interbody spacer of FIG. 4A;

FIG. 5A illustrates a top perspective view of an interbody spacer or porous regions of the interbody spacer, according to embodiments of the present disclosure;

FIG. 5B illustrates a bottom perspective view of the interbody spacer or porous regions of the interbody spacer of FIG. 5A;

FIG. 5C illustrates a front view of the interbody spacer or porous regions of the interbody spacer of FIG. 5A;

FIG. 5D illustrates a rear view of the interbody spacer or porous regions of the interbody spacer of FIG. 5A;

FIG. 5E illustrates a left side view of the interbody spacer or porous regions of the interbody spacer of FIG. 5A;

FIG. 5F illustrates a right side view of the interbody spacer or porous regions of the interbody spacer of FIG. 5A;

FIG. 5G illustrates a top view of the interbody spacer or porous regions of the interbody spacer of FIG. 5A;

FIG. 5H illustrates a bottom view of the interbody spacer or porous regions of the interbody spacer of FIG. 5A;

FIG. 6 illustrates a top perspective view of an integrally formed interbody spacer, according to embodiments of the present disclosure;

FIG. 7 illustrates a bottom perspective view of an inserter, according to an embodiment of the present disclosure;

FIG. 8 illustrates a top perspective view of the inserter of FIG. 7;

FIG. 9 illustrates a bottom view of the inserter of FIG. 7;

FIG. 10 illustrates a top view of the inserter of FIG. 7;

FIG. 11 illustrates a top perspective view of a distal end of the inserter of FIG. 7;

FIG. 12 illustrates a bottom perspective view the distal end of the inserter of FIG. 7;

FIG. 13 illustrates a perspective view of the inserter of FIG. 7 coupling with the implant assembly of FIG. 2;

FIG. 14 illustrates a perspective view of the inserter and implant assembly from FIG. 13 coupled together;

FIG. 15 illustrates a perspective view of a distal end of the system shown in FIG. 14 with inferior and superior fasteners;

FIG. 16 illustrates a perspective view of the system shown in FIG. 15 with the inferior and superior fasteners installed in the implant assembly;

FIG. 17 illustrates a perspective view of the system shown in FIG. 16 with the inserter decoupled from the implant assembly for removal;

FIG. 18 illustrates a perspective view of a locking plate coupling with the implant assembly, according to an embodiment of the present disclosure;

FIG. 19 illustrates a perspective view of the locking plate coupled to the implant assembly;

FIG. 20 illustrates a cross-sectional top view of the locking plate and implant assembly from FIG. 19; and

FIG. 21 illustrates a perspective view of the implant assembly installed within an intervertebral space of a spine.

It is to be understood that the drawings are for purposes of illustrating the concepts of the present disclosure and may not be drawn to scale. Furthermore, the drawings illustrate exemplary embodiments and do not represent limitations to the scope of the present disclosure.

### DETAILED DESCRIPTION

Exemplary embodiments of the present disclosure will be best understood by reference to the drawings, wherein like parts are designated by like numerals throughout. It will be readily understood that the components of the present disclosure, as generally described and illustrated in the drawings, could be arranged, and designed in a wide variety of different configurations. Thus, the following more detailed description of the embodiments of the devices, systems, methods, and instruments, as represented in the drawings, is not intended to limit the scope of the present disclosure but is merely representative of exemplary embodiments of the present disclosure.

The word “exemplary” is used herein to mean “serving as an example, instance, or illustration.” Any embodiment described herein as “exemplary” is not necessarily to be construed as preferred or advantageous over other embodiments. While the various aspects of the embodiments are presented in the drawings, the drawings are not necessarily drawn to scale unless specifically indicated.

Standard medical planes of reference and descriptive terminology are employed in this specification. While these terms are commonly used to refer to the human body, certain terms may also be applicable to physical objects in general.

A standard system of three mutually perpendicular reference planes is employed. A sagittal plane divides a body into right and left portions. A coronal plane divides a body into anterior and posterior portions. A transverse plane divides a body into superior and inferior portions. A mid-sagittal, mid-coronal, or mid-transverse plane divides a body into equal portions, which may be bilaterally symmetric. The intersection of the sagittal and coronal planes defines a superior-inferior or cephalad-caudal axis. The intersection of the sagittal and transverse planes defines an anterior-posterior axis. The intersection of the coronal and transverse planes defines a medial-lateral axis. The superior-inferior or cephalad-caudal axis, the anterior-posterior axis, and the medial-lateral axis are mutually perpendicular.

Anterior means toward the front of a body. Posterior means toward the back of a body. Superior or cephalad means toward the head. Inferior or caudal means toward the feet or tail. Medial means toward the midline of a body, particularly toward a plane of bilateral symmetry of the body. Lateral means away from the midline of a body or away from a plane of bilateral symmetry of the body. Axial means toward a central axis of a body. Abaxial means away from a central axis of a body. Ipsilateral means on the same side of the body. Contralateral means on the opposite side of the body. Proximal means toward the trunk of the body. Proximal may also mean toward a user or operator. Distal means away from the trunk. Distal may also mean away from a user or operator. Dorsal means toward the top of the foot. Plantar means toward the sole of the foot. Varus means outboard deviation of the knees (away from the sagittal plane) from the line between the hip and ankle, resulting in a “bowlegged” stance. Valgus means inboard deviation of the knees (toward the sagittal plane) from the line between the hip and ankle, resulting in a “knock-kneed” stance.

Although the implants, systems, methods, and instruments described herein are disclosed in the context of spinal fusions, it will be readily understood by those of skill in the art that the inventive concepts described and contemplated herein may be applied to any implant, system, method, or instrument for any bone or joint of a patient or an animal, including, but not limited to, bones/joints of the foot, ankle, knee, hip, hand, wrist, elbow, shoulder, sacral, neck, etc., without departing from the spirit or scope of the present disclosure.

It will also be understood that any component or feature of any implant, system, method, or instrument described or contemplated herein may be combined with any other component or feature that is described or contemplated herein to create any number of different implant, system, method, or instrument embodiments.

It will also be understood that any component or feature of any implant, system, method, or instrument described or contemplated herein may be combined with any other component or feature that is described or contemplated herein to create any number of different surgical kits. Each of these surgical kits may also include (or not include) any number of supporting surgical instruments or general parts (e.g., K-wires, drill bits, rasps, trialing instruments, bone screws, implants with different sizes, etc.).

Moreover, it will also be understood that any method step (or component/feature of any method step) that is described or contemplated herein may be combined with any other method step (or component/feature of any method step) that is described or contemplated herein in any order, and/or in any combination, to create any number of different method embodiments for implanting any number of different implant or system embodiments described or contemplated herein.

As used herein, the term “additive manufacturing process” may include any manufacturing process that creates one or more physical objects by joining materials to create a physical object (e.g., layering one or more materials on top of each other, etc.). An additive manufacturing process may include, but is not limited to, a process or technique that utilizes 3D printing, porous surface deposition, nano-coating, stereolithography, VAT photopolymerization, selective or direct laser sintering/melting, electron beam sintering/melting, fused deposition modeling, material jetting, binder jetting, material extrusion, powder bed fusion, sheet lamination, directed energy deposition, etc.

As used herein, the term “subtractive manufacturing process” may include any manufacturing process that creates one or more physical objects by removing one or more materials or portions of materials from a physical object. A subtractive manufacturing process may include, but is not limited to, a process or technique that utilizes machining (e.g., cutting, boring, drilling, grinding, milling, reaming, etc.), casting, molding, bone extraction/shaping, electrical discharge machining (EDM), laser cutting, plasma cutting, water jet cutting, etc.

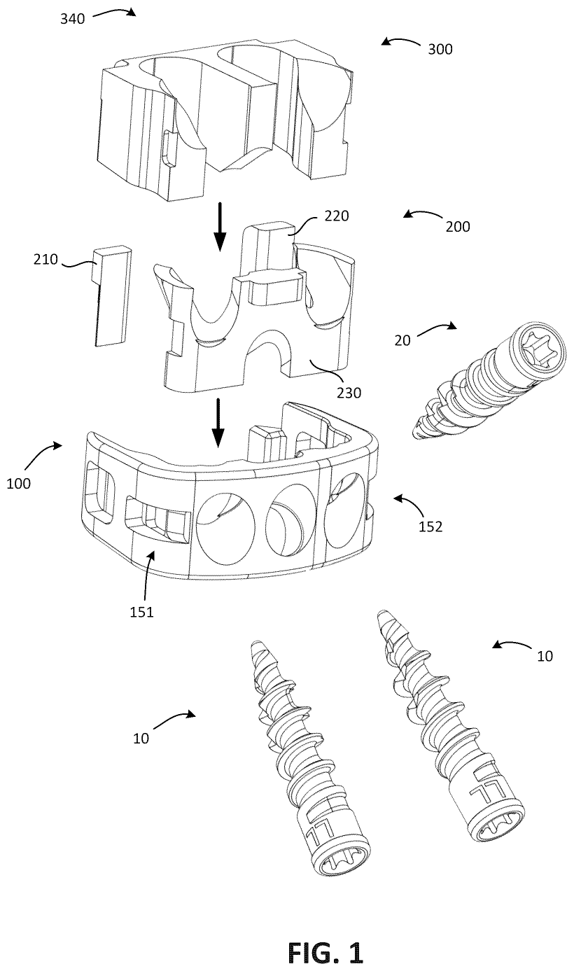

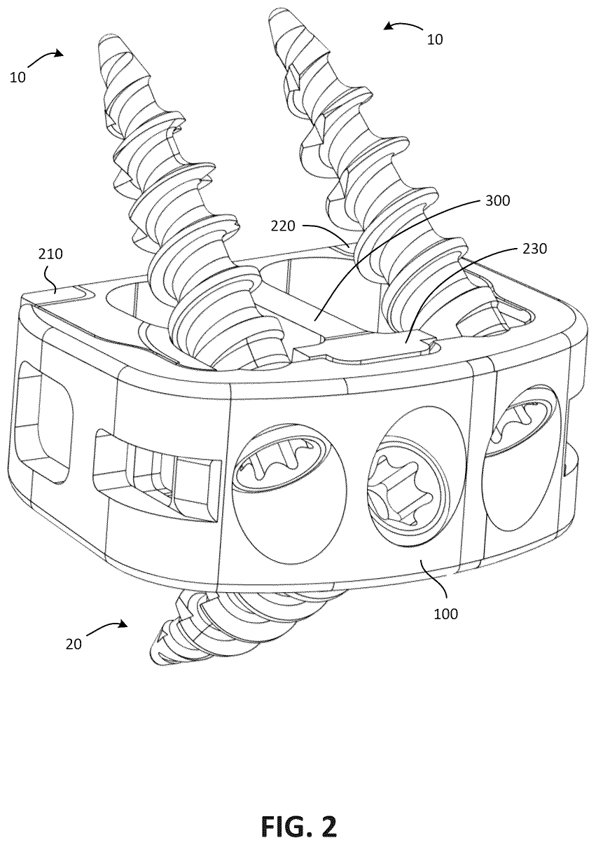

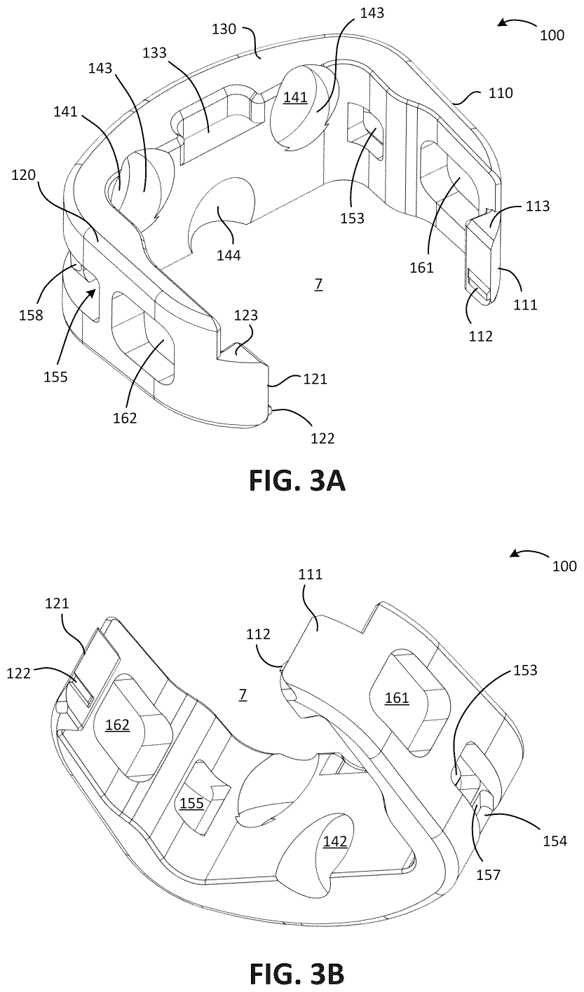

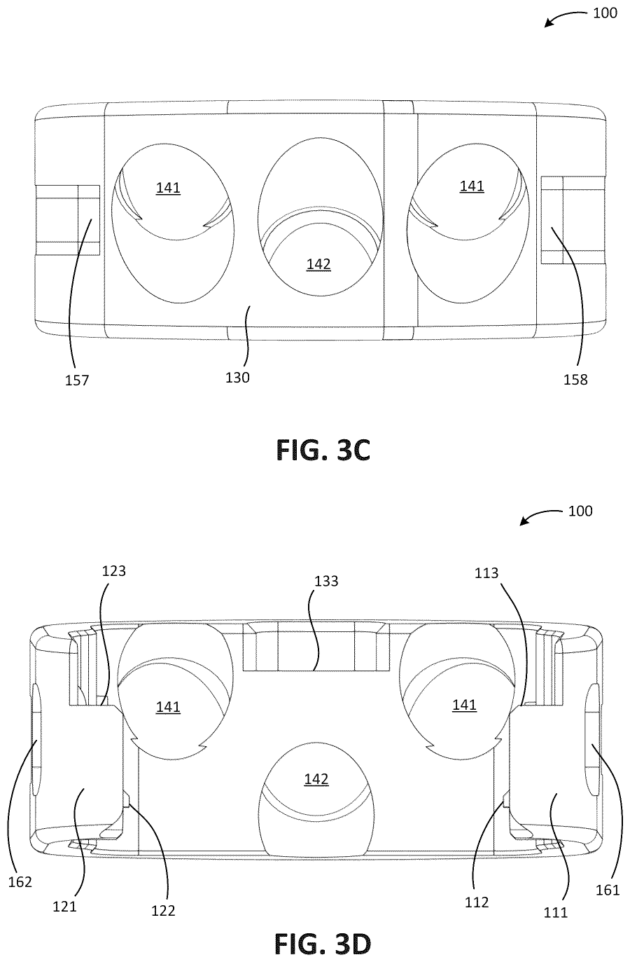

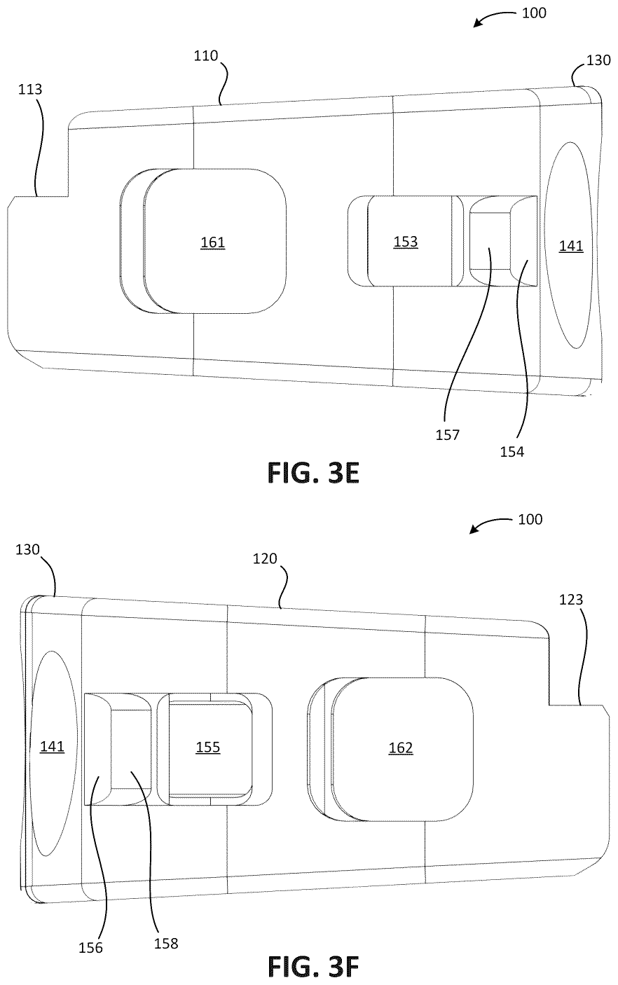

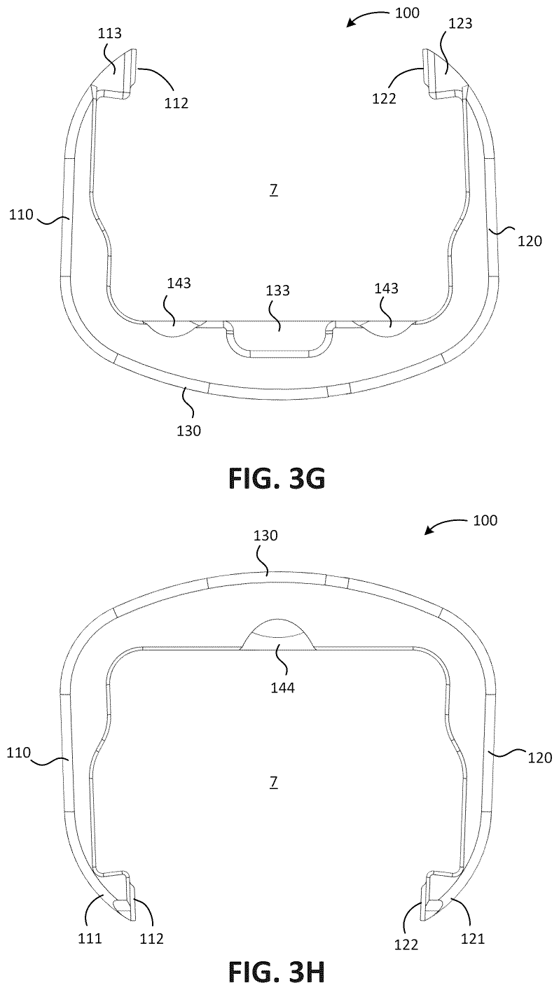

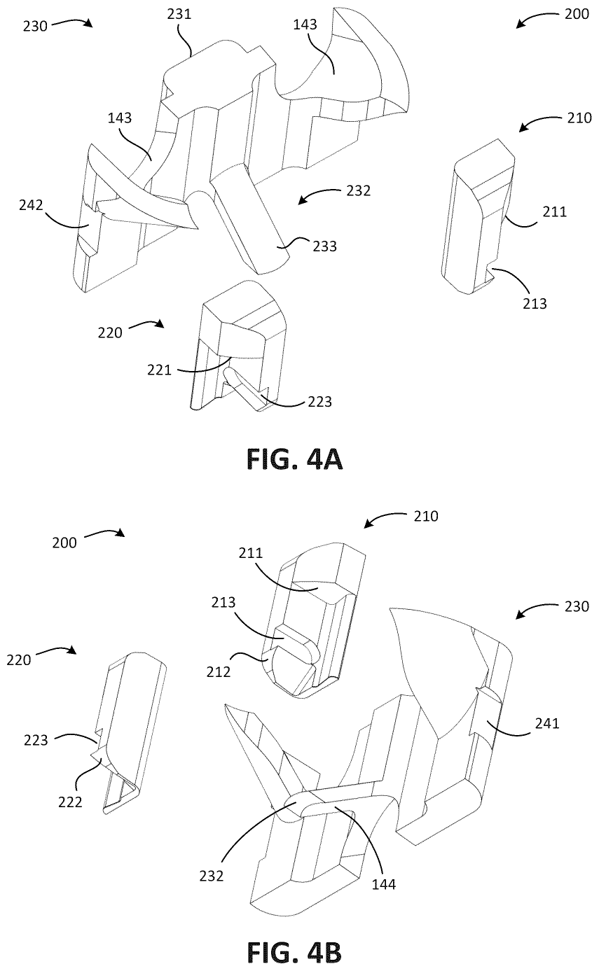

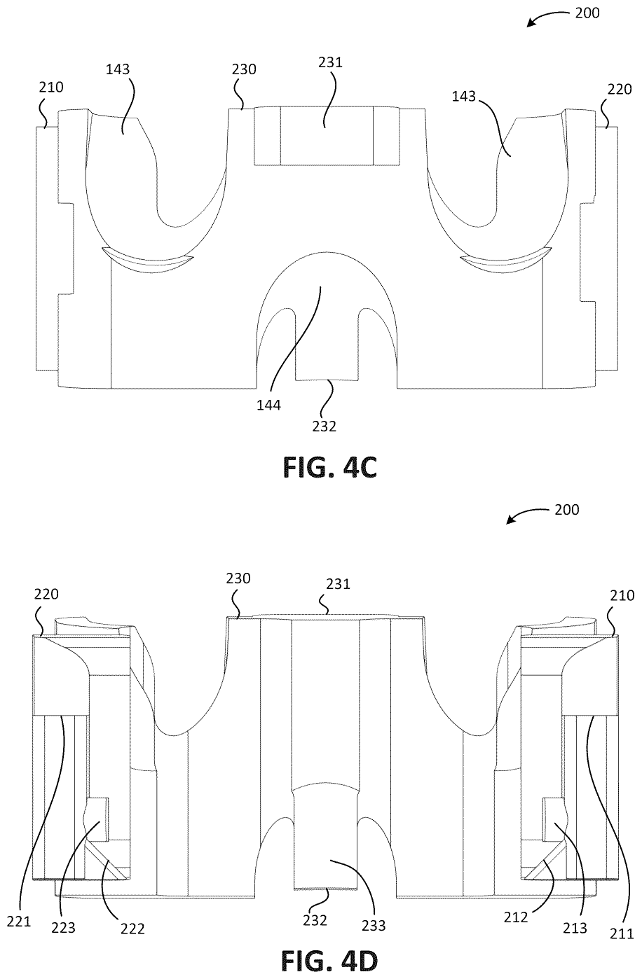

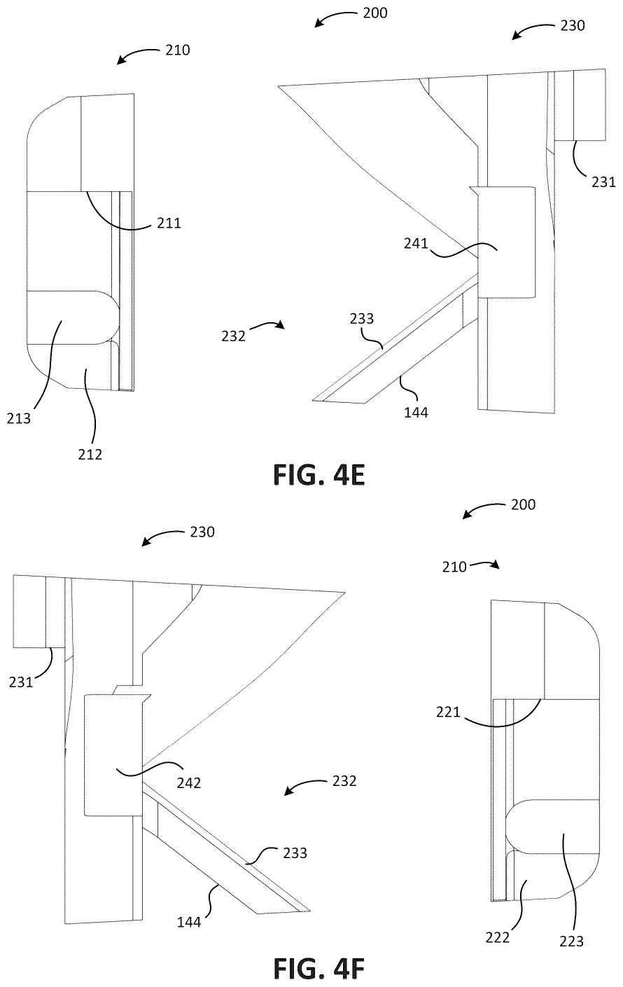

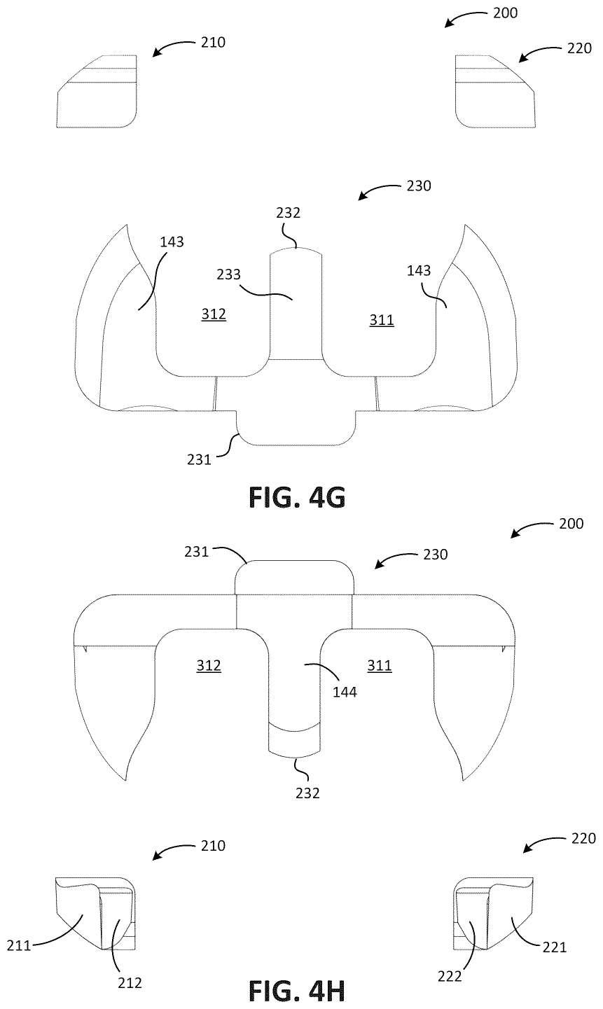

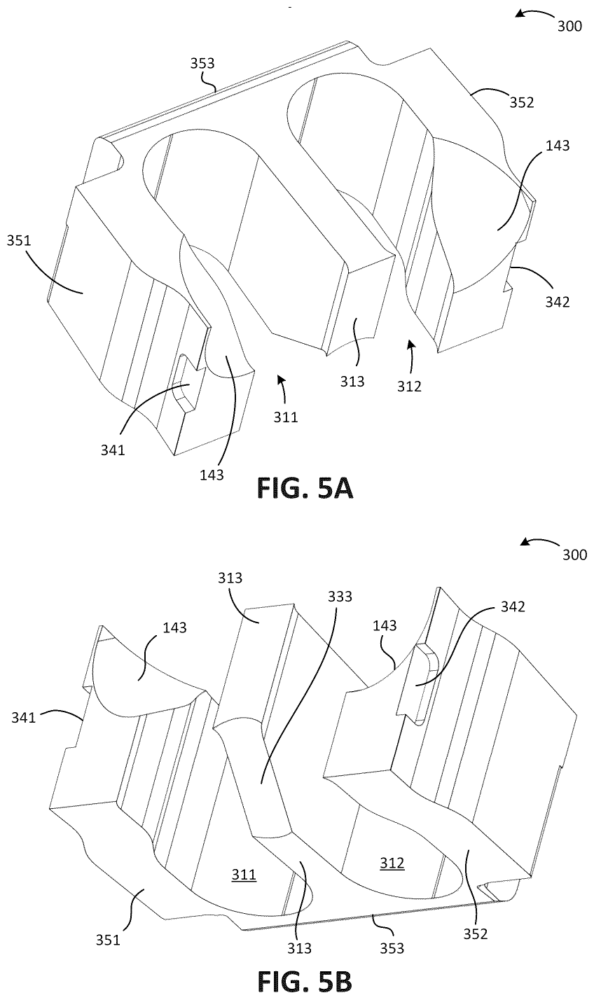

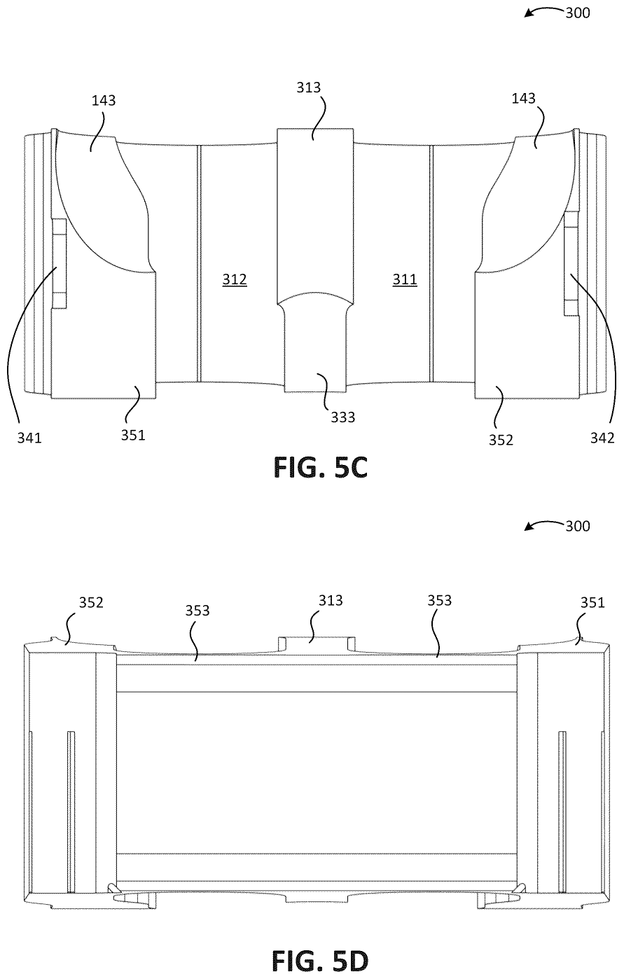

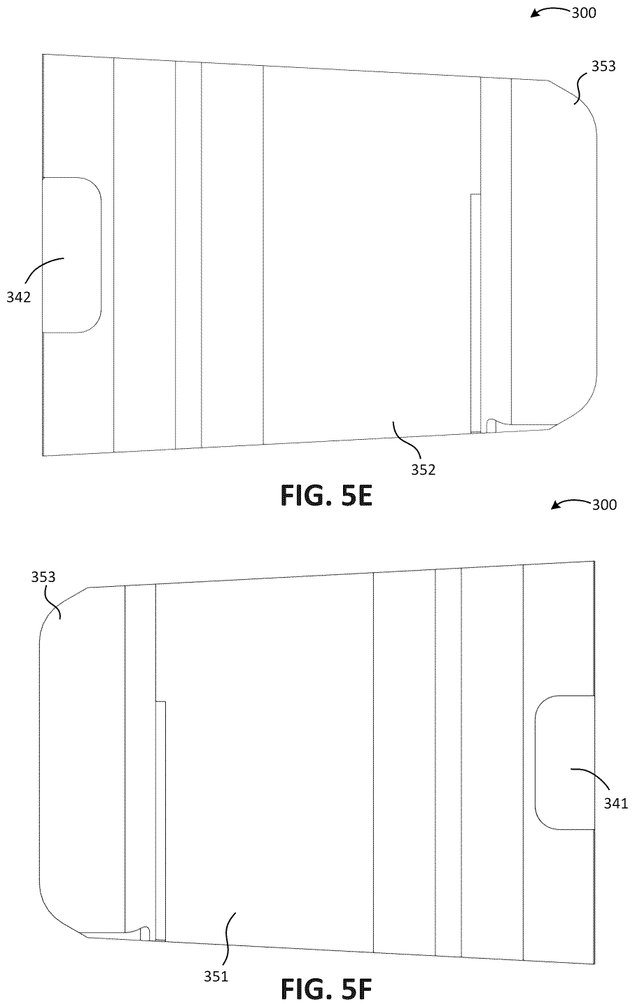

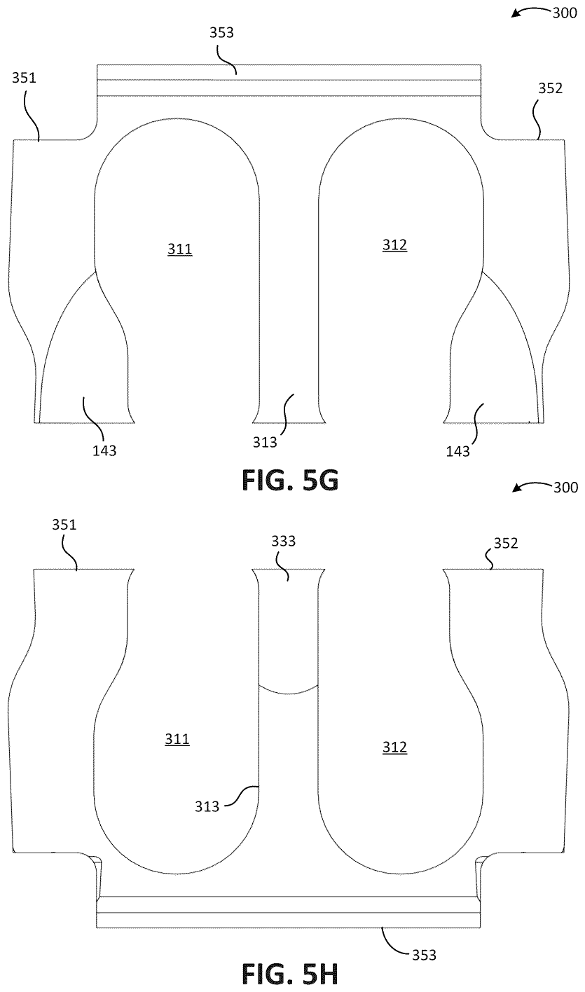

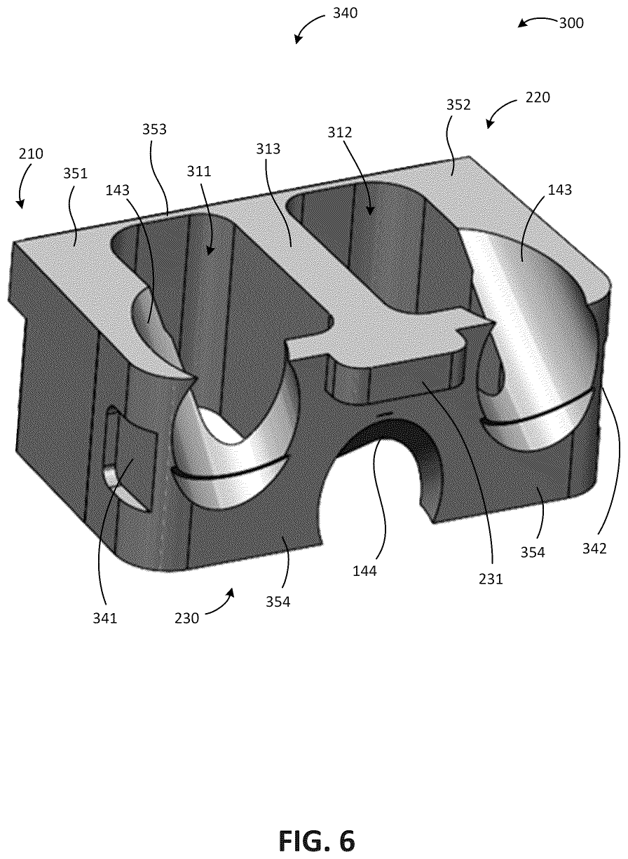

FIGS. 1-6 illustrate various views of implant systems or assemblies for stabilizing a spinal joint, according to embodiments of the present disclosure. Specifically, FIG. 1 shows an exploded view of an implant assembly; FIG. 2 shows the implant assembly of FIG. 1 assembled together; FIGS. 3A-3H show various views of a bone plate 100 of the implant assembly; FIGS. 4A-4H show various views of solid regions of an interbody spacer, a spacer insert assembly, or a spacer insert 200 of the implant assembly, according to embodiments of the present disclosure; FIGS. 5A-5H show various views of an interbody spacer 300 or a porous region 340 of the interbody spacer 300 of the implant assembly, according to embodiments of the present disclosure; and FIG. 6 shows an integrally formed interbody spacer 300 of the implant assembly that may include at least one porous region and/or at least one solid region, according to embodiments of the present disclosure.

As shown in FIG. 6, in at least some embodiments the interbody spacer 300 may be integrally formed as a single piece and may comprise at least one porous region, a central porous region, or the porous region 340 of the interbody spacer 300, which may be formed via at least one additive manufacturing process. The interbody spacer 300 may also include at least one solid region formed via at least one additive manufacturing process. For example, the interbody spacer 300 shown of FIG. 6 may be integrally formed as a single piece including: (1) the interbody spacer 300 or porous region 340 of the interbody spacer 300; (2) a first solid region of the interbody spacer 300 or first spacer insert 210; (3) a second solid region of the interbody spacer 300 or second spacer insert 220; and (4) a third solid region of the interbody spacer 300 or third spacer insert 230. However, it will be understood that the interbody spacer 300 may comprise any number of porous regions and/or solid regions. In this manner, the porous region 340 of the interbody spacer 300 may facilitate bony in-growth to aid the bone fusion/healing process, and the one or more solid regions of the interbody spacer 300 may help stabilize the intervertebral space during the bone fusion/healing process by providing additional structural integrity that maintains the intervertebral space during the bone fusion/healing process.

In some embodiments, the implant assembly (or assemblies) shown in FIGS. 1-6 may be assembled by inserting the interbody spacer 300 (and/or the spacer insert 200, if utilized as a separate piece in some embodiments) into a cavity 7 of the bone plate 100 that is formed between a first arm 110 and a second arm 120 of the bone plate 100. The first arm 110 and the second arm 120 of the bone plate 100 may extend opposite each other from opposing ends of an anterior member 130 of the bone plate 100 to form the cavity 7 between the first arm 110, the second arm 120, and the anterior member 130.

In some embodiments, the interbody spacer 300 (and/or the spacer insert 200, if utilized as a separate piece in some embodiments) may be inserted into the cavity 7 of the bone plate 100 from a superior direction. However, it will be understood that the interbody spacer 300 (and/or the spacer insert 200, if utilized as a separate piece in some embodiments) may be inserted into the bone plate 100 from an inferior direction and/or from a posterior direction.

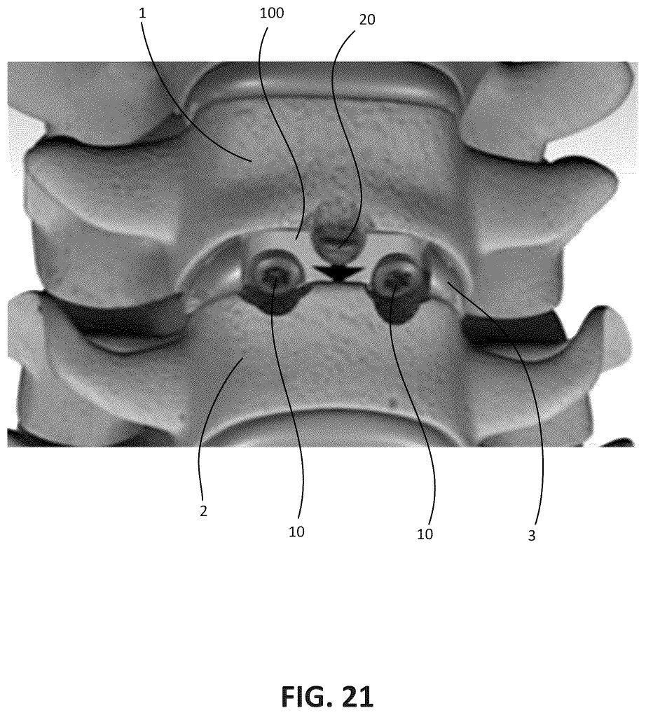

In some embodiments, the implant assemblies of FIGS. 1-6 may be installed within an intervertebral space 3 between a first or superior vertebral body 1, and a second or inferior vertebral body 2 (e.g., see FIG. 21), as will be discussed below in more detail.

In some embodiments, the implant assemblies of FIGS. 1-6 may be assembled within the intervertebral space 3 between the superior vertebral body 1 and the inferior vertebral body 2 by: (a) inserting the interbody spacer 300 (and/or the spacer insert 200, if utilized as a separate piece in some embodiments) into the intervertebral space 3; and then (b) inserting the bone plate 100 into the intervertebral space 3 and around the interbody spacer 300 (and/or around the spacer insert 200, if utilized as a separate piece in some embodiments) in order to assemble the implant assembly within the intervertebral space 3.

In some embodiments, the implant assemblies of FIGS. 1-6 may then be secured to the superior vertebral body 1 and/or the inferior vertebral body 2 by: (c) inserting at least one superior fastener (or a superior fastener 10) into at least one superior fastener aperture (or a superior fastener aperture 141) that is formed through the anterior member 130 of the bone plate 100 and into the superior vertebral body 1; and (d) inserting at least one inferior fastener (or an inferior fastener 20) into at least one inferior fastener aperture (or an inferior fastener aperture 142) that is formed through the anterior member 130 of the bone plate 100 and into the inferior vertebral body 2.

In some embodiments, the superior fastener 10 may be guided along a superior trajectory into the superior vertebral body 1 via at least one superior ramp surface (or a superior ramp 143) forming a first superior angle with respect to the anterior member 130 of the bone plate 100, and the inferior fastener 20 may be guided along an inferior trajectory into the inferior vertebral body 2 via at least one inferior ramp surface (or an inferior ramp 144) forming a second inferior angle with respect to the anterior member 130 of the bone plate 100.

In some embodiments, the superior ramp 143 and/or the inferior ramp 144 may each extend from the bone plate 100 to the spacer insert 200 and/or the interbody spacer 300 to help guide the superior fastener 10 into the superior vertebral body 1, and/or help guide the inferior fastener 20 into the inferior vertebral body 2.

In some embodiments, the implant assembly may comprise the spacer insert 200 and the interbody spacer 300 as separate pieces, which are shown in FIGS. 1-2 and 4A-5H. In these embodiments, the spacer insert 200 and the interbody spacer 300 from FIGS. 1-2 and 4A-5H may each be inserted into the cavity 7 of the bone plate 100 one at a time, or together.

In some embodiments, the spacer insert 200 may comprise one or more spacer inserts such as the first spacer insert 210, the second spacer insert 220, and the third spacer insert 230. However, it will be understood that the spacer insert 200 may comprise any number of separate spacer insert pieces, or no separate pieces at all. Moreover, it will be understood that the interbody spacer 300, the first spacer insert 210, the second spacer insert 220, and the third spacer insert 230 may all be integrally formed together as one single piece, as previously discussed above with reference to FIG. 6.

In some embodiments, the first spacer insert 210 may include a first spacer tab 211 configured to abut against a first arm stop surface 113 of a first arm projection 111 that extends medially from a distal end of the first arm 110 toward the second arm 120 to help position the first spacer insert 210 relative to the bone plate 100.

In some embodiments, the first spacer insert 210 may also include a first spacer guide surface 212 configured to slidingly engage a first arm projection tab 112 extending from the first arm projection 111 to guide the first arm projection tab 112 into a first spacer recess 213 formed in the first spacer insert 210 to secure the first spacer insert 210 to the first arm 110 of the bone plate 100.

In some embodiments, the second spacer insert 220 may include a second spacer tab 221 configured to abut against a second arm stop surface 123 on a second arm projection 121 that extends medially from a distal end of the second arm 120 toward the first arm 110 to help position the second spacer insert 220 relative to the bone plate 100.

In some embodiments, the second spacer insert 220 may also include a second spacer guide surface 222 configured to slidingly engage a second arm projection tab 122 extending from the second arm projection 121 to guide the second arm projection tab 122 into a second spacer recess 223 formed in the second spacer insert 220 to secure the second spacer insert 220 to the second arm 120 of the bone plate 100.

In some embodiments, the third spacer insert 230 may include a third spacer tab 231 configured to abut against a third stop surface 133 on the anterior member 130 of the bone plate 100 to help position the third spacer insert 230 relative to the bone plate 100.

In some embodiments, the third spacer insert 230 may also include a posterior projection member 232 comprising the inferior ramp 144 surface on an inferior side of the posterior projection member 232, and a posterior projection member stop surface 233 on a superior side of the posterior projection member 232. In these embodiments, the posterior projection member stop surface 233 may abut against an interbody spacer stop surface 333 on an inferior surface of the interbody spacer 300 to help position the interbody spacer 300 relative to the bone plate 100.

In some embodiments, the first spacer insert 210, the second spacer insert 220, the third spacer insert 230 (or solid regions of the integrally formed interbody spacer), and/or the bone plate 100 may be formed from a durable/strong material, such as a metal, a metal alloy, a durable plastic, etc., (e.g., titanium, a titanium alloy or other metal alloy such as cobalt chromium, UHMWPE, etc.). However, it will be understood that all or any portion of the spacer insert 200 or bone plate 100 may comprise any material suitable for implantation within a patient (e.g., plastics, PEEK, hydroxyapatite, bone graft material, allograft material, etc.).

In some embodiments, all or any portion of the spacer insert 200 and/or bone plate 100 may be formed a subtractive manufacturing process, such as a machining process, a casting process, a molding process, a bone extraction/shaping process, etc. However, it will be understood that all or any portion of the spacer insert 200 (or solid regions of the integrally formed interbody spacer) and/or the bone plate 100 can also be formed via an additive manufacturing process or 3D printing process as well.

In some embodiments, the interbody spacer 300, the spacer insert 200, and/or the bone plate 100 may be formed from a porous material and/or any other material that may comprise a surface coating to facilitate bony ingrowth/fusion (e.g., a surface coating that may be applied to any material via a porous surface deposition process, such as a nano coating process, etc.). However, it will also be understood that the interbody spacer 300, the spacer insert 200, and/or the bone plate 100 may be formed from any material that may be suitable for implantation within a patient (e.g., metals such as titanium, metal alloys such as cobalt chromium, UHMWPE, plastics, PEEK, hydroxyapatite, bone graft material, allograft material, etc.).

In some embodiments, the interbody spacer 300, the spacer insert 200, and/or the bone plate 100 may be formed via a 3D printing or additive manufacturing process. However, it will be understood that all or any portion of the interbody spacer 300, the spacer insert 200, and/or the bone plate 100 may be formed via a subtractive manufacturing process, a machining process, a casting process, a molding process, a bone extraction/shaping process, etc. For example, in some embodiments the spacer insert 200 may be formed via a subtractive manufacturing process, such as a machining process, and the interbody spacer 300 may be formed via an additive manufacturing process, such as a 3D printing process, etc.

As previously discussed, in some embodiments the implant assembly may comprise the interbody spacer 300 that is shown in FIG. 6 (instead of the interbody spacer 300 and the spacer insert 200 that are shown in FIGS. 1-2 and 4A-5H).

The interbody spacer 300 of FIG. 6 may be formed of any material(s) and/or via any manufacturing processes discussed above with respect to the interbody spacer 300, the bone plate 100, and/or the spacer insert 200 shown in FIGS. 1-5H.

The interbody spacer 300 of FIG. 6 may also be inserted into the cavity 7 of the bone plate 100 from a superior direction, an inferior direction, and/or a posterior direction.

In some embodiments, the interbody spacer 300 of FIG. 6 may be formed to incorporate one or more (or all) of the features of the first spacer insert 210, the second spacer insert 220, and/or the third spacer insert 230 into the interbody spacer 300 itself, as shown in FIG. 6.

In some embodiments, the interbody spacer 300 may include a first spacer cavity 311 and/or a second spacer cavity 312 that may be separated from each other by a partition member 313 between a first spacer wall 351, a second spacer wall 352, a posterior spacer wall 353, and/or an anterior spacer wall 354 of the interbody spacer 300.

In some embodiments, the first spacer cavity 311 and/or the second spacer cavity 312 may be configured to receive a bone fusion material therein (e.g., a bone cement, a bone graft material, etc.) to help facilitate the bone fusion process.

In some embodiments, the first arm 110 of the bone plate 100 may include a first arm window 161 formed therethrough, and the second arm 120 of the bone plate 100 may include a second arm window 162 formed therethrough.

In some embodiments, the bone plate 100 may include a first inserter connection feature 151 and a second inserter connection feature 152.

In some embodiments, the first inserter connection feature 151 may include a first connection window 153, a first connection recess 154, and a first connection feature guide surface 157 intermediate the first connection recess 154 and the first connection window 153 which may be configured to guide a first inserter finger 431 of an inserter tool or inserter 400 into the first connection window 153.

In some embodiments, the first inserter connection feature 151 may also include a first insert connection recess 241 formed in the third spacer insert 230, and/or a first spacer connection recess 341 formed in the interbody spacer 300.

In some embodiments, the second inserter connection feature 152 may include a second connection window 155, a second connection recess 156, and a second connection feature guide surface 158 intermediate the second connection recess 156 and the second connection window 155 that may be configured to guide a second inserter finger 432 of the inserter 400 into the second connection window 155.

In some embodiments, the second inserter connection feature 152 may also include a second insert connection recess 242 formed in the third spacer insert 230, and/or a second spacer connection recess 342 formed in the interbody spacer 300.

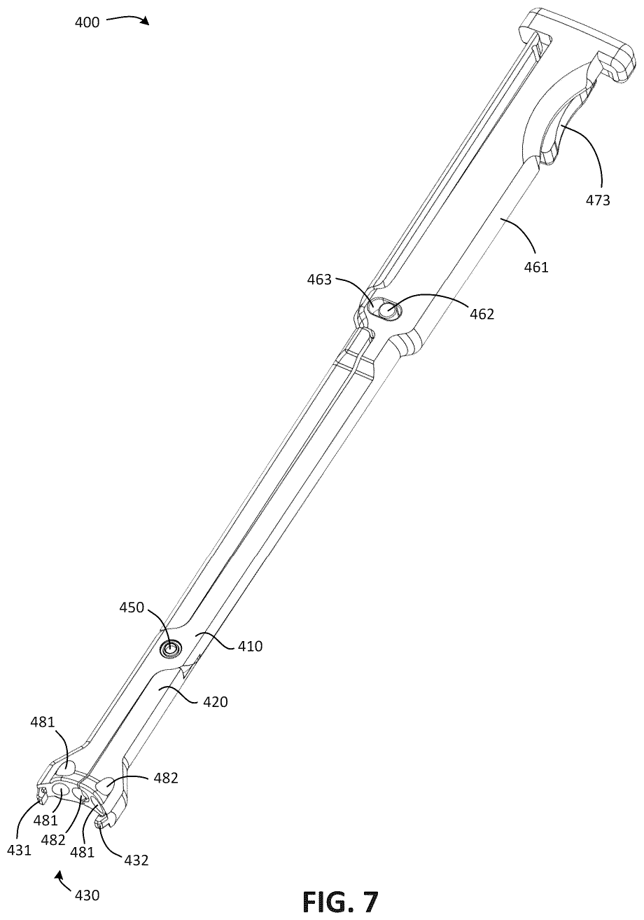

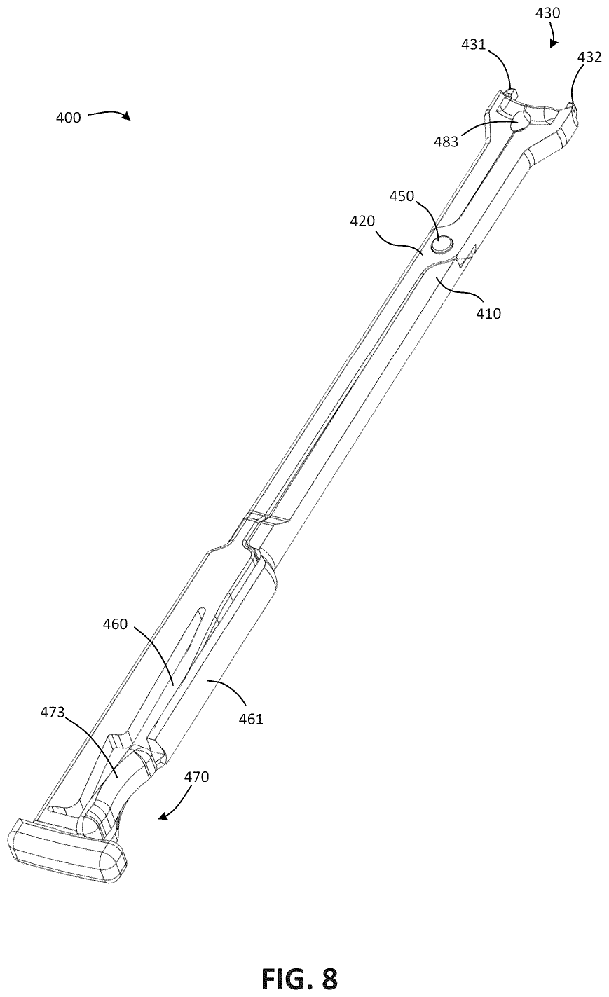

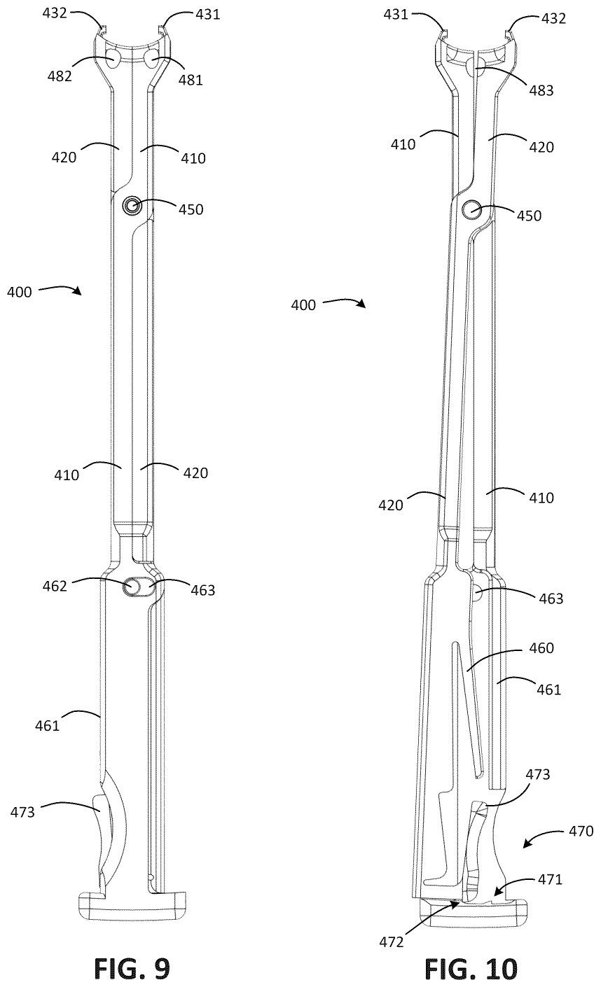

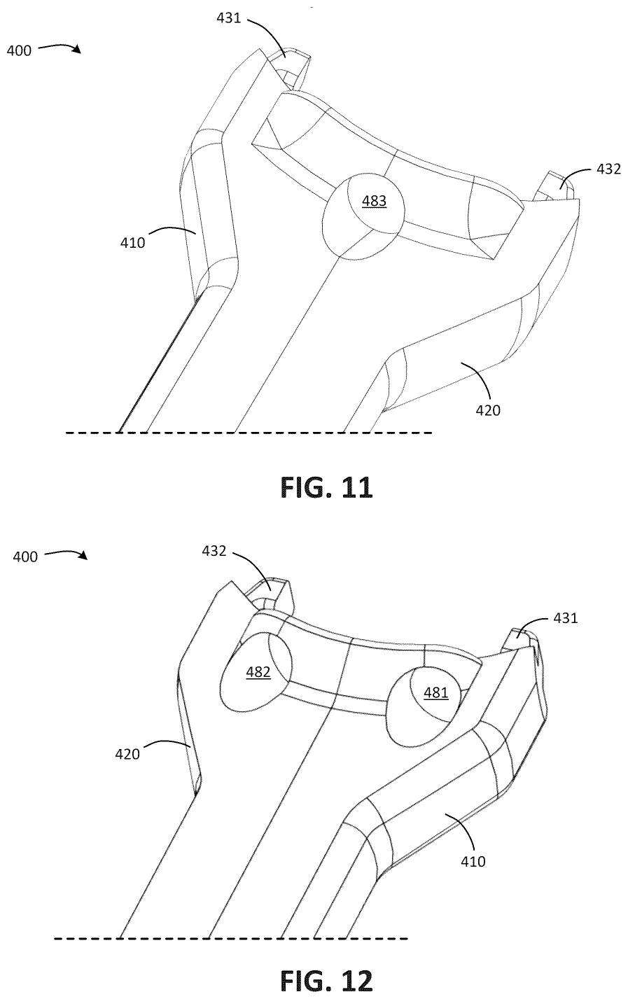

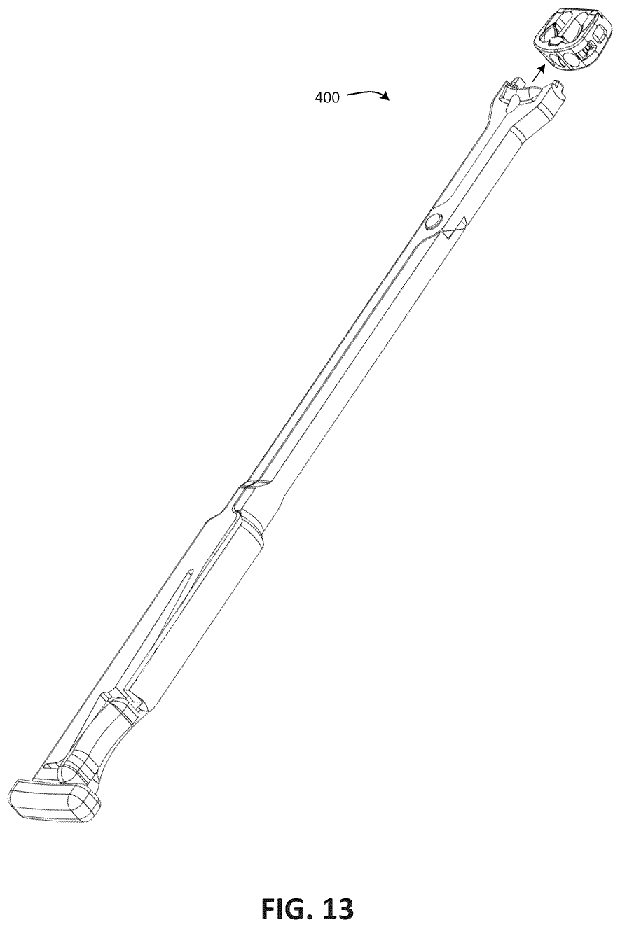

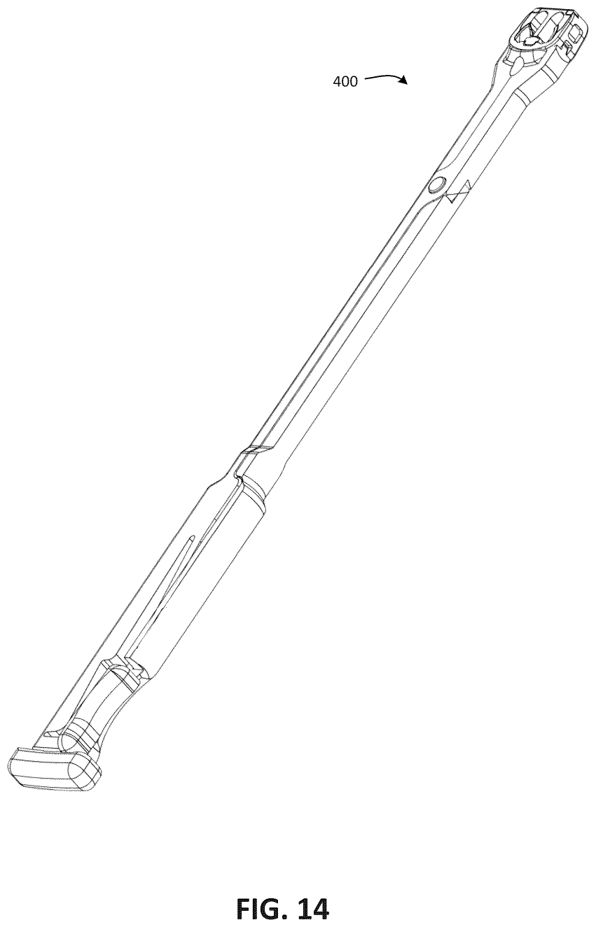

FIGS. 7-12 illustrate various views of the inserter 400 instrument for inserting the implant assemblies described or contemplated herein, according to an embodiment of the present disclosure. Specifically, FIG. 7 shows a bottom perspective view of the inserter 400; FIG. 8 shows a top perspective view of the inserter 400; FIG. 9 shows a bottom view of the inserter 400; FIG. 10 shows a top view of the inserter 400; FIG. 11 shows a close up top view of a distal end of the inserter 400; and FIG. 12 shows a close up bottom view of the distal end of the inserter 400.

In some embodiments, the inserter 400 may generally include a first inserter arm 410 pivotally coupled with a second inserter arm 420 via a pivot pin 450. This configuration may allow a proximal end of the first inserter arm 410 to pivot toward or away from a proximal end of the second inserter arm 420, causing a distal end of the first inserter arm 410 to likewise pivot toward or away from a distal end of the second inserter arm 420. For example, when the proximal end of the first inserter arm 410 pivots away from the proximal end of the second inserter arm 420 (e.g., see FIG. 10), the distal end of the first inserter arm 410 will likewise pivot away from the distal end of the second inserter arm 420 to open up an inserter connection feature 430 located at the distal end of the inserter 400 for receiving the implant assembly therein. The proximal end of the first inserter arm 410 may then be pivoted toward the proximal end of the second inserter arm 420 (e.g., see FIG. 9), causing the distal end of the first inserter arm 410 to likewise pivot toward the distal end of the second inserter arm 420 to engage the inserter connection feature 430 with the bone plate 100 connection feature and couple the implant assembly to the inserter 400.

In some embodiments, the inserter connection feature 430 may include the first inserter finger 431 disposed at the distal end of the first inserter arm 410 configured to be received within the first connection window 153 formed in the bone plate 100, as well as the second inserter finger 432 disposed at the distal end of the second inserter arm 420 configured to be received within the second connection window 155 formed in the bone plate 100 to couple the implant assembly to the inserter 400.

In some embodiments, the proximal ends of the first inserter arm 410 and the second inserter arm 420 may be pivotally biased away from each other via a resilient inserter member 460 formed on the second inserter arm 420 that may push against a rigid member 461 formed on the first inserter arm 410.

In some embodiments, the proximal ends of the first inserter arm 410 and the second inserter arm 420 may be prevented from pivoting too far away from each other via a stop pin 462 coupled to the second inserter arm 420 that may project into a stop slot 463 formed in the first inserter arm 410.

In some embodiments, the proximal ends of the first inserter arm 410 and the second inserter arm 420 may be pivoted toward each other to couple the implant assembly to the inserter 400, and this coupled engagement between the implant assembly and the inserter 400 may be held in place in a first locked configuration of the inserter 400. For example, in some embodiments the inserter 400 may include an inserter locking feature 470 at the proximal end of the inserter 400. The inserter locking feature 470 may include a locking tab 471 projecting from the first inserter arm 410 that may engage with a lock surface 472 formed on a resilient locking member 473 coupled to the second inserter arm 420. In this manner, the proximal ends of the first inserter arm 410 and the second inserter arm 420 may be pivoted toward each to engage the lock surface 472 against the locking tab 471 to hold the inserter 400 in the first locked configuration and maintain the coupled engagement of the implant assembly with the inserter 400 (e.g., see FIG. 14) to facilitate insertion of the implant assembly into the patient.

Once the implant assembly has been installed in the patient, the inserter locking feature 470 may then be disengaged to decouple the implant assembly from the inserter 400 in a second unlocked configuration. This may be accomplished by pressing the resilient locking member 473 inward to disengage the lock surface 472 on the resilient locking member 473 from the locking tab 471 on the first inserter arm 410. The proximal ends of the first inserter arm 410 and the second inserter arm 420 will then be free to pivot away from each other, causing the distal ends of the first inserter arm 410 and the second inserter arm 420 to also pivot away from each other to decouple the implant assembly from the inserter 400 in the second unlocked configuration.

In some embodiments, the distal ends of the first inserter arm 410 and/or the second inserter arm 420 may include at least one superior guide hole formed therethrough and configured to guide the superior fastener 10 into the superior vertebral body 1 (e.g., see FIG. 12). For example, in some embodiments a first superior guide hole 481 may be formed through the distal end of the first inserter arm 410, and a second superior guide hole 482 may be formed through the distal end of the second inserter arm 420.

In some embodiments, the distal ends of at least one of the first inserter arm 410 and/or the second inserter arm 420 may also include at least one inferior guide hole 483 formed therethrough and configured to guide the inferior fastener 20 into inferior vertebral body 2 (e.g., see FIG. 11).

FIGS. 13-20 illustrate various views of a method for installing the implant assemblies described herein.

FIG. 13 shows the inserter 400 coupling with the implant assembly. As previously described, the inserter 400 may be placed in the second unlocked configuration to open up the inserter locking feature 470 at the distal end of the inserter 400 to couple with the implant assembly.

FIG. 14 shows the inserter 400 and implant assembly coupled together, as previously described. This may be accomplished by placing the inserter 400 in the first locked configuration to maintain the coupled engagement of the implant assembly to the inserter 400 and facilitate insertion of the implant assembly into a patient.

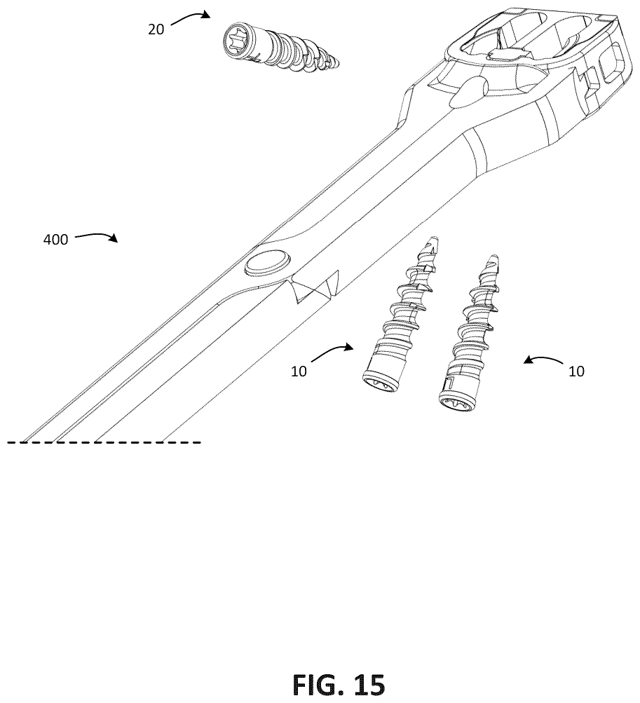

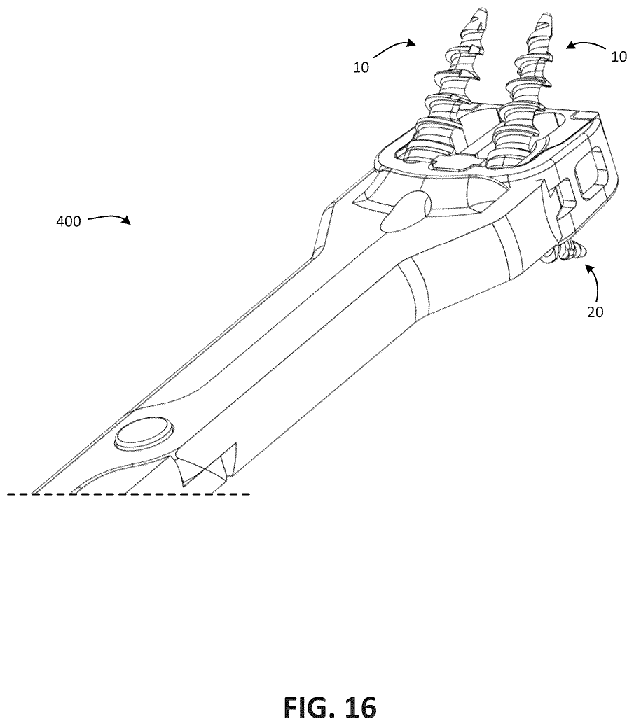

FIG. 15 shows the distal end of the inserter 400 and implant assembly construct with inferior and superior fasteners oriented for installation into the implant assembly. This may be accomplished with the inferior and superior guide holes that are formed in the distal ends of the first inserter arm 410 and the second inserter arm 420, as previously described. FIG. 16 shows the distal end of the inserter 400 and implant assembly construct with the inferior and superior fasteners installed in the implant assembly.

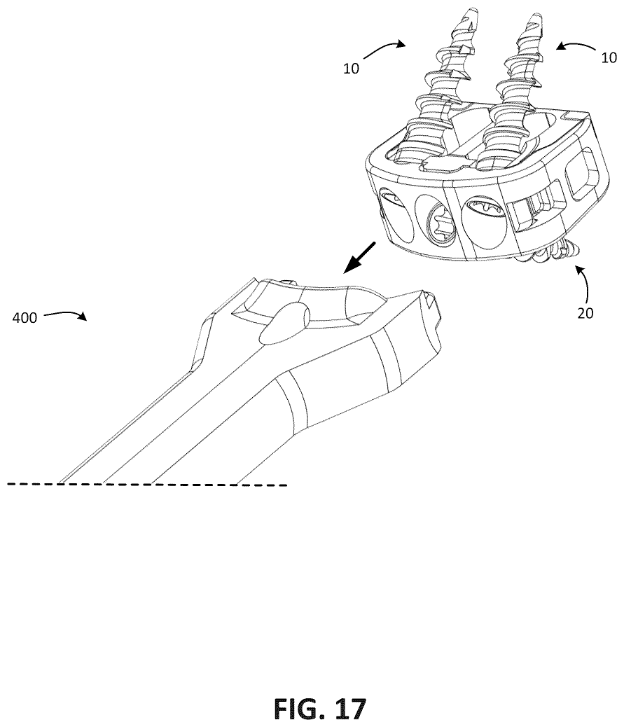

FIG. 17 shows the distal end of the inserter 400 and implant assembly construct with the inserter 400 placed in the second unlocked configuration to decouple the implant assembly from the inserter 400, allowing the inserter 400 to be removed from the implantation site within the patient after the implant assembly has been installed within the intervertebral space 3 of the patient (e.g., see FIG. 21).

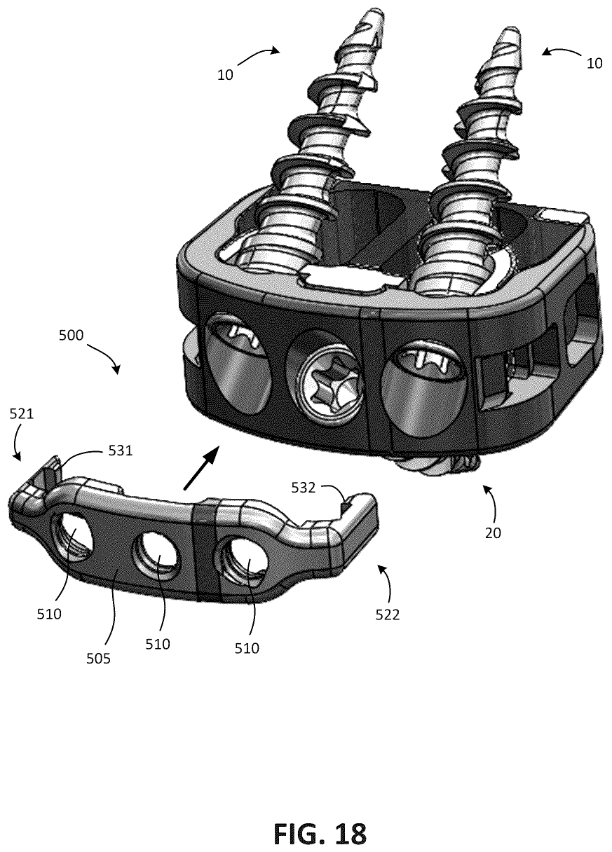

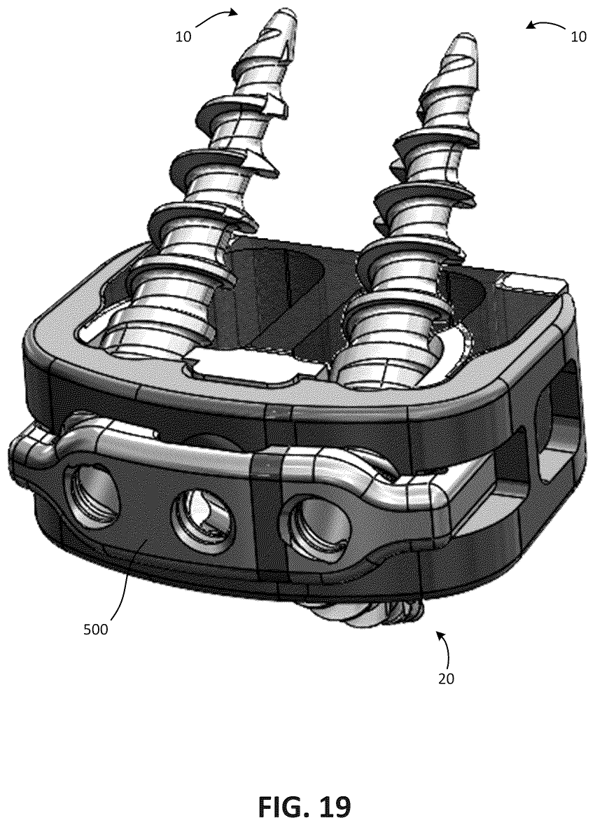

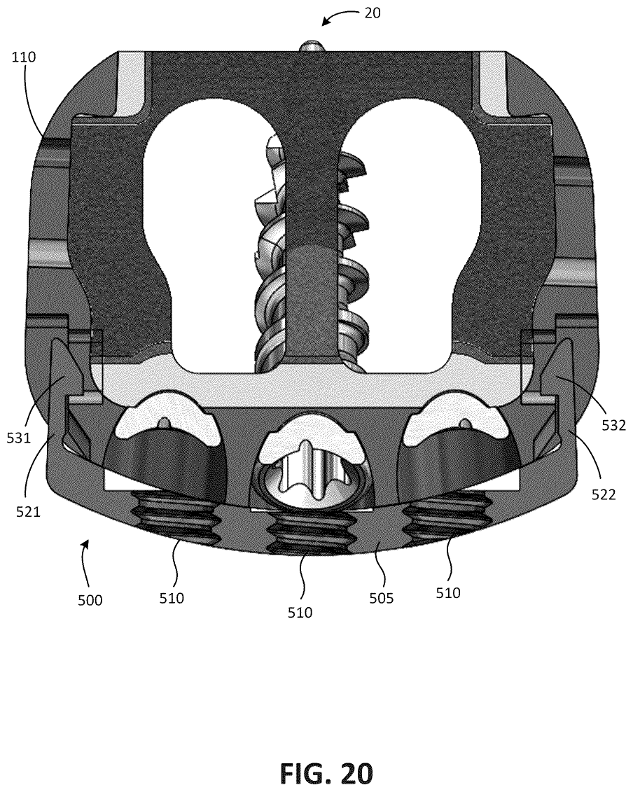

FIGS. 18-20 show a locking plate 500 that may be utilized with the implant assemblies describe herein, according to an embodiment of the present disclosure. Specifically, FIG. 18 shows the locking plate 500 coupling with the implant assembly, FIG. 19 shows the locking plate 500 coupled to the implant assembly, and FIG. 20 shows a cross-sectional top view of the locking plate 500 coupled to implant assembly.

In some embodiments, the locking plate 500 may be configured to prevent the superior and/or inferior fasteners from backing out of the implant assembly over time during the healing process.

In some embodiments, the locking plate 500 may generally include a locking plate body 505, one or more threaded apertures or a threaded aperture 510 formed through the locking plate body 505, a first locking plate arm 521 extending from a first end of the locking plate 500, and a second locking plate arm 522 extending from a second end of the locking plate 500.

In some embodiments, the threaded aperture 510 may be configured to couple with a locking plate inserter tool (not shown) which may be configured to insert the locking plate 500 into the patient and/or couple the locking plate 500 to the implant assembly.

In some embodiments, the threaded aperture 510 may also be configured to receive one or more threaded set screws therein (not shown) which may be configured to abut against the heads of the superior and/or inferior fasteners to provide additional support against backing out during the healing process.

In some embodiments, the first locking plate arm 521 may include a first locking plate arm tab 531 configured to slide along the first connection feature guide surface 157 and resiliently snap into the first connection window 153 during insertion, and the second locking plate arm 522 may include a second locking plate arm tab 532 configured to slide along the second connection feature guide surface 158 and resiliently snap into the second connection window 155 to couple the locking plate 500 to the implant assembly and prevent the superior and/or inferior fasteners from backing out of the implant assembly over time during the healing process (e.g., see FIGS. 19 and 20).

Any procedures or methods disclosed herein may comprise one or more steps or actions for performing the described method. The method steps and/or actions may be interchanged with one another. In other words, unless a specific order of steps or actions is required for proper operation of the embodiment, the order and/or use of specific steps and/or actions may be modified.

Reference throughout this specification to “an embodiment” or “the embodiment” means that a particular feature, structure, or characteristic described in connection with that embodiment is included in at least one embodiment. Thus, the quoted phrases, or variations thereof, as recited throughout this specification are not necessarily all referring to the same embodiment.

Similarly, it should be appreciated that in the above description of embodiments, various features are sometimes grouped together in a single embodiment, figure, or description thereof for the purpose of streamlining the present disclosure. This method of disclosure, however, is not to be interpreted as reflecting an intention that any embodiment requires more features than those expressly recited in that embodiment. Rather, inventive aspects lie in a combination of fewer than all features of any single foregoing disclosed embodiment.

Recitation of the term “first” with respect to a feature or element does not necessarily imply the existence of a second or additional such feature or element. Elements recited in means-plus-function format are intended to be construed in accordance with 35 U.S.C. § 112. It will be apparent to those having skill in the art that changes may be made to the details of the above-described embodiments without departing from the underlying principles set forth herein. The phrases “connected to”, “coupled to”, “engaged with”, and “in communication with” may refer to any form of interaction between two or more entities, including mechanical, electrical, magnetic, electromagnetic, fluid, and thermal interaction. Two components may be functionally coupled to each other even though they are not in direct contact with each other. The term “coupled” can include components that are coupled to each other via integral formation, components that are removably and/or non-removably coupled with each other, components that are functionally coupled to each other through one or more intermediary components, etc. The term “abutting” refers to items that may be in direct physical contact with each other, although the items may not necessarily be attached together. The phrase “fluid communication” refers to two or more features that are connected such that a fluid within one feature is able to pass into another feature. As defined herein the term “substantially” means within +/−20% of a target value, measurement, or desired characteristic.

While specific embodiments and applications of the present disclosure have been illustrated and described, it is to be understood that the scope of the present disclosure is not limited to the precise configurations and components disclosed herein. Various modifications, changes, and variations which will be apparent to those skilled in the art may be made in the arrangement, operation, and details of the devices, systems, and methods disclosed herein.

What is claimed is:

1. An implant assembly for stabilizing a joint between a superior vertebra and an inferior vertebra, the implant assembly comprising:
a bone plate comprising:
an anterior member comprising:
at least one superior fastener aperture formed therein and configured to receive at least one superior fastener therethrough; and
at least one inferior fastener aperture formed therein and configured to receive at least one inferior fastener therethrough;
a first arm extending from a first end of the anterior member; and
a second arm extending from a second end of the anterior member, opposite the first arm, to form a cavity intermediate the first arm and the second arm of the bone plate; and
an interbody spacer configured for placement within the cavity of the bone plate;
wherein:
at least a portion of the interbody spacer is formed via at least one additive manufacturing process; and
at least a portion of the bone plate is formed via at least one subtractive manufacturing process.

2. The implant assembly of claim 1, wherein the at least one additive manufacturing process comprises at least one of:
a 3D printing process;
a porous surface deposition process; and
a nano coating process.

3. The implant assembly of claim 1, wherein the at least one subtractive manufacturing process comprises at least one of:
a machining process;
a casting process;
a molding process;
a bone extraction process; and
a bone shaping process.

4. The implant assembly of claim 1, wherein:
the interbody spacer comprises at least one porous region formed via a 3D printing process; and
the bone plate comprises at least one durable material formed via a machining process.

5. The implant assembly of claim 4, wherein:
the interbody spacer further comprises at least one solid region formed via the 3D printing process.

6. The implant assembly of claim 1, wherein the interbody spacer is configured to be inserted into the cavity of the bone plate from at least one of:
a superior direction;
an inferior direction; and
a posterior direction.

7. The implant assembly of claim 1, further comprising:
a locking plate couplable with the bone plate,
wherein the locking plate is configured to prevent the at least one superior fastener and the at least one inferior fastener from backing out of the implant assembly.

8. An implant assembly for stabilizing a joint between a superior vertebra and an inferior vertebra, the implant assembly comprising:
a bone plate comprising:
an anterior member comprising:
at least one superior fastener aperture formed therein and configured to receive at least one superior fastener therethrough; and
at least one inferior fastener aperture formed therein and configured to receive at least one inferior fastener therethrough;
a first arm extending from a first end of the anterior member; and
a second arm extending from a second end of the anterior member, opposite the first arm, to form a cavity of the bone plate intermediate the first arm and the second arm of the bone plate; and
an interbody spacer configured for placement within the cavity of the bone plate, the interbody spacer comprising:
at least one porous region formed via at least one additive manufacturing process; and
at least one solid region formed via the at least one additive manufacturing process.

9. The implant assembly of claim 8, wherein:
the at least one porous region comprises:
a central porous region; and
the at least one solid region comprises:
a first solid region;
a second solid region; and
a third solid region.

10. The implant assembly of claim 8, wherein the at least one additive manufacturing process comprises at least one of:
a 3D printing process;
a porous surface deposition process; and
a nano coating process.

11. The implant assembly of claim 8, wherein:
at least a portion of the bone plate is formed via at least one subtractive manufacturing process.

12. The implant assembly of claim 11, wherein the at least one subtractive manufacturing process comprises at least one of:
a machining process;
a casting process;
a molding process;
a bone extraction process; and
a bone shaping process.

13. The implant assembly of claim 8, wherein the interbody spacer is configured to be inserted into the cavity of the bone plate from at least one of:
a superior direction;
an inferior direction; and
a posterior direction.

14. The implant assembly of claim 8, further comprising:
a locking plate couplable with the bone plate,
wherein the locking plate is configured to prevent the at least one superior fastener and the at least one inferior fastener from backing out of the implant assembly.

15. An inserter for implanting an implant assembly into an intervertebral joint space between a superior vertebra and an inferior vertebra, the inserter comprising
a first inserter arm comprising:
a proximal end;
a distal end; and
a first inserter arm connection feature disposed on the distal end of the first inserter arm;
a second inserter arm comprising:
a proximal end;
a distal end; and
a second inserter arm connection feature disposed on the distal end of the second inserter arm; and
a pivot pin configured to pivotally couple the first inserter arm to the second inserter arm;
wherein:
in a first unlocked configuration, the proximal end of the first inserter arm is pivoted away from the proximal end of the second inserter arm to pivot the distal end of the first inserter arm away from the distal end of the second inserter arm to receive the implant assembly between the first inserter arm connection feature and the second inserter arm connection feature; and
in a second locked configuration, the proximal end of the first inserter arm is pivoted toward the proximal end of the second inserter arm to pivot the distal end of the first inserter arm toward the distal end of the second inserter arm to couple the implant assembly between the first inserter arm connection feature and the second inserter arm connection feature of the inserter.

16. The inserter of claim 15, further comprising:
a rigid member formed on the first inserter arm; and
a resilient inserter member formed on the second inserter arm,
wherein the resilient inserter member is configured to push against the rigid member to pivotally bias the proximal end of the first inserter arm away from the proximal end of the second inserter arm.

17. The inserter of claim 16, further comprising:
a locking tab projecting from the first inserter arm; and
a lock surface formed on a resilient locking member coupled to the second inserter arm,
wherein, the lock surface formed on the resilient locking member is configured to engage the locking tab projecting from the first inserter arm and hold the inserter in the second locked configuration.

18. The inserter of claim 15, further comprising at least one inferior guide hole formed through at least one of:
the distal end of the first inserter arm; and
the distal end of the second inserter arm.

19. The inserter of claim 15, further comprising at least one superior guide hole formed through at least one of:
the distal end of the first inserter arm; and
the distal end of the second inserter arm.

20. The inserter of claim 19, wherein the at least one superior guide hole comprises:
a first superior guide hole formed through the distal end of the first inserter arm; and
a second superior guide hole formed through the distal end of the second inserter arm.
